(19) United States
(12) Patent Application Publication
(10) Pub. No.: US 2019/0167542 A1
(43) Pub. Date: Jun. 6, 2019

(54) KIT FOR THE COSMETIC TREATMENT OF THE EYE AND THE SKIN, AND COSMETIC FOR APPLICATION TO THE EYE AND TO THE SKIN

(71) Applicant: Ursapharm Arzneimittel GmbH, Saarbrücken (DE)

(72) Inventors: Markus Mahler, Völklingen (DE); Ute Steinfeld, St. Ingbert (DE); Dominik Holzer, St. Ingbert (DE)

(21) Appl. No.: 16/131,744

(22) PCT Filed: Mar. 17, 2017
(86) PCT No.: PCT/EP2017/056390

§ 371 Date: Sep. 14, 2018

(30) Foreign Application Priority Data
Mar. 17, 2016 (DE) 10 2016 204 472.9

(51) Int. Cl. A61K 8/19; A61K 8/73; A61K 8/368; A61K 8/34; A61K 8/9789; A61K 8/58; A61K 8/365; A61Q 19/10; A45D 44/00; A45D 34/04

(52) U.S. Cl. CPC A61K 8/19; A45D 2200/155; A61K 8/368; A61K 8/345; A61K 8/9789; A61K 8/58; A61K 8/365; A61Q 19/10; A45D 44/002; A45D 34/04; A61K 2800/30; A61K 2800/87; A45D 2200/25; A45D 2200/1036; A61K 8/735

(57) ABSTRACT

The invention relates to a kit for topical, cosmetic treatment of the eye and of the skin and cosmetic contained therein for application on the eye and on the skin.

The invention relates to a kit for topical, cosmetic treatment of the eye and of the skin and a cosmetic contained therein for application on the eye and on the skin.

Cosmetic formulations for application on the eye, which are known from this state of the art, are similar, in their composition, to the natural film on the eye surface. This means that they comprise both aqueous and oily components. One disadvantage of these already known compositions is frequently that they comprise interface-active substances (emulsifiers) in order to be able to ensure long-lasting dispersion and storage life.

Emulsifiers have an amphiphilic molecule structure, consisting of a polar, hydrophilic and a non-polar, lipophilic molecule part which are spatially separated from each other. They reduce the interface tension between the phases by being disposed on the interface between both liquids and form an interface film. Frequently emulsifier mixtures are also used for stabilisation of emulsions.

Conventional emulsifiers can be subdivided, corresponding to their hydrophilic molecule part, into ionic (anionic, cationic and amphoteric) and non-ionic: the most well-known example of an anionic emulsifier is soap, as the water-soluble sodium- or potassium salts of the saturated and unsaturated higher fatty acids are usually termed. Important representatives of the cationic emulsifiers are quaternary ammonium compounds. The hydrophilic molecule part of non-ionic emulsifiers frequently consists of glycerine, polyglycerine, sorbitans, carbohydrates or polyoxyethylene glycols and is generally linked to the lipophilic molecule part via ester and ether bonds. The latter normally consists of fatty alcohols, fatty acids or isofatty acids. By varying the structure and the size of the polar and of the non-polar molecule part, the lipophily and hydrophily of emulsifiers can be changed within broad limits.

Although the use of these emulsifiers in pharmaceutical, cosmetic or dermatological preparations per se is safe, emulsifiers can cause allergic reactions in individual cases. Particularly in the case of eyes which are irritated anyway, such additives can lead to intolerances and further irritations, because emulsifiers impair the tear film.

In addition to emulsifiers, cosmetic formulations for application on the eye typically also comprise preservatives in order to avoid microbial contamination by bacteria, yeasts and fungi. Frequently, benzalkonium chloride, benzododecinium chloride, cetrimide, chlorohexidine, chlorobutanol, sodium hydroxymethylglycinate, sodium perborate, phosphonic acid, phenoxyethanol, phenylmercuric acetate, polyhexanide, polyquat, purites and thiomersal are added as preservative. Furthermore, further preservatives are benzoic acid, propionic acid, hexadienoic acid, formaldehyde, inorganic sulphites, 2-phenoxyethanol, benzyl alcohol, hexamethylene tetramine and chloroacetamide.

Even for the listed preservatives, it is not unusual for them to trigger intolerances, in particular allergies and further dermatological reactions. Benzalkonium chloride, benzododecinium chloride and cetrimide reduce for example the stability of the tear film and, with long-term application, cause a dry eye. They attack the cornea of the eye so that the uppermost corneal layer becomes thinner at some places and corneal ulcers can occur. Chlorohexidine leads, on the other hand, to allergic reactions.

Starting from the state of the art, it was therefore the object of the present invention to provide a cosmetic for application on the eye which is based on an aqueous solution and the storage life and stability of which can be ensured without the addition of emulsifiers or preservatives. In addition, a kit is intended to be made available which makes cosmetic treatment of the eye with the corresponding cosmetic possible for the user in a simple manner and without special previous experience.

This object is achieved by a cosmetic having the features of patent claim 1, which is contained in a kit having the features of patent claim 12. Patent claim 16 shows in addition the use of the kit. Advantageous embodiments and developments are contained in the dependent claims.

The cosmetic according to the invention for application on the eye and on the skin comprises or consists of an aqueous solution which comprises hyaluronic acid or a salt of hyaluronic acid, salicylic acid or a salt of salicylic acid, sorbitol, Euphrasia mother tincture and a buffer system or consists hereof, no emulsifiers being added to the solution.

Preferably, the cosmetic in addition does not include any preservative, in particular no preservative selected from the group consisting of benzoic acid, propionic acid, hexadienoic acid, formaldehyde, inorganic sulphites, 2-phenoxyethanol, benzyl alcohol, hexamethylene tetramine, chloroacetamide, benzalkonium chloride, benzododecinium chloride, cetrimide, chlorohexidine, chlorobutanol, sodium hydroxymethylglycinate, sodium perborate, phosphonic acid, phenylmercuric acetate, polyhexanide, polyquad, purites, thiomersal and mixtures hereof. This is therefore advantageous because many preservatives develop a cornea-toxic effect. Preservative-free cosmetics can, in contrast, be applied above all by contact lens wearers and those suffering from allergies during the entire pollen season without risk.

In the cosmetic according to the invention, salicylic acid does not fulfil the function of a preservative but effects cleaning and care of the eyelids and parts of the eye and assists disintegration of glutinous and encrusted deposits on the eye and in addition has a locally soothing and pain-relieving effect.

In addition, it is advantageous if the cosmetic or the solution forming the base of the cosmetic is phosphate-free. There are understood, by phosphates, in the sense according to the invention, all phosphate-comprising ions, such as for example dihydrogen phosphates, hydrogen phosphates, phosphates, but also di-, tri- or metaphosphates. Freedom from phosphates is provided if the cosmetic or the solution forming the base comprises no phosphate ions, i.e. no phosphate ions can be detected in the cosmetic by means of current analytical methods.

According to a particularly preferred embodiment, the cosmetic is preservative- and phosphate-free.

Preferably, the aqueous solution has a pH value in the range of 6.0 to 8.0, preferably in the range of 7.0 to 7.2.

Furthermore, it is advantageous if the cosmetic comprises salicylic acid or a salt of salicylic acid in a weight proportion of 0.01 to 2.00% by weight, preferably of 0.01 to 0.3% by weight, in particular of 0.1% by weight. In this concentration, the salicylic acid has an anti-inflammatory and pain-relieving effect.

Furthermore, it is preferred if the solution comprises hyaluronic acid or a sodium salt of hyaluronic acid in a weight proportion of 0.01 to 1.00% by weight, particularly preferably of 0.01 to 0.03% by weight, in particular of 0.02% by weight. This protects the eye from drying-out due to its highly hydrophilic properties.

In an advantageous variant of the invention, the solution comprises sorbitol in a weight proportion of 1.0 to 4.0% by weight, preferably of 2.8% by weight. This is used as osmotic and contributes to the isotonicity of the solution.

Furthermore, it is preferred that the solution comprises Euphrasia mother tincture in a weight proportion of 0.001 to 0.4% by weight, preferably of 0.3% by weight.

Euphrasia mother tincture thereby represents a plant extract. An extract can be obtained, according to the invention, from plants or parts thereof, for example using water. Extracts can furthermore be liquid or solid in nature.

Preferably, the Euphrasia mother tincture is obtained from fresh or dried above-ground parts of Euphrasia officinalis, such as leaves, flowers and/or stems. Extracts of the entire plant of Euphrasia officinalis, also termed herba Euphrasiae, are particularly preferred.

There is understood by “Euphrasia” (eyebright) in the sense of this invention, all types of the Euphrasia genus normally indicated under this term, for example Euphrasia stricta, Euphrasia montana and/or Euphrasia rostkoviana.

There is understood by the term “mother tincture” in the sense of this invention, extracts from plants or parts thereof which are produced using solvents, such as water and/or alcohols, such as ethanol, plant extracts or solutions of dried plants in a solvent mixture, such as an alcohol-water mixture.

In addition, it is advantageous if the cosmetic according to the present invention or the solution forming the base comprises at least one buffer system, selected from the group consisting of citrate buffer, borate buffer, phosphate, tris buffer, trometamol/maleic acid buffer or mixtures or combinations hereof. In the case where the cosmetic is intended to be configured preferably phosphate-free, as described previously, the presence of a phosphate buffer must of course be dispensed with.

It can be provided that the cosmetic comprises a buffer system comprising or consisting of
sodium citrate or a hydrate of sodium citrate, preferably in a weight proportion of 0.2 to 1.5% by weight, particularly preferably of 0.5 to 1.0% by weight, in particular of 0.9% by weight,
citric acid, preferably in a weight proportion of 0.002 to 0.008% by weight, particularly preferably of 0.002% by weight and
NaOH.

With the help of this composition of the buffer system, it can be ensured that the cosmetic has a physiological pH value.

As an alternative hereto, it can be provided that the solution or the cosmetic comprises no buffer system.

A preferred embodiment of the cosmetic consists of a solution which comprises a sodium salt of hyaluronic acid in a proportion of 0.01 to 1.0% by weight, particularly preferably of 0.01 to 0.03% by weight, in particular of 0.02% by weight, salicylic acid in a proportion of 0.01 to 2.00% by weight, particularly preferably of 0.01 to 0.03% by weight, in particular of 0.02% by weight, sorbitol in a proportion of 0.5 to 5.0% by weight, in particular of 2.80% by weight, sodium citrate dihydrate in a proportion of 0.25 to 2.50% by weight, in particular of 0.85% by weight and Euphrasia mother tincture in a proportion of 0.05 to 1.50% by weight, in particular of 0.30% by weight, and has a pH value of 7.0 to 7.2.

The cosmetic or the solution forming the base can be applied directly for example on the lid for cosmetic purposes but also indirectly by applying the cosmetic on a cotton wool pad or a paper tissue and the lid being moistened or cleaned therewith.

The kit according to the invention for cosmetic treatment of the eye comprises the above-described cosmetic and also, separately from the cosmetic, at least a cotton wool pad, a paper tissue and/or an eye mask.

The kit is thereby distinguished preferably by the eye mask being heatable and particularly preferably comprising a latent heat reservoir, in particular a sodium acetate trihydrate-containing latent heat reservoir.

Furthermore, the cotton wool pad, the cosmetic tissue and/or the eye mask can be configured to receive the cosmetic or the eye mask can be configured to receive the cotton wool pad.

The cosmetic or the solution forming the base of the cosmetic can be stored in the previously described kit in a multi-dosage system, a disposable dosage system or in paper tissues which are moistened with the cosmetic. A multi-dosage system can comprise for example a storage container with a pump head, by means of which repeatedly single dosages of the cosmetic can be dispensed. A disposable dosage system comprises a single, separately packed portion of the cosmetic. The moist tissues can thereby be hygienic and sterile, for example it can be provided that single ones or several of the moist tissues are sealed separately in a bag. Hence ease of use and also long storage life can be ensured.

The moist tissues can thereby be used alone but also in combination with the previously mentioned eye mask and hence also as an alternative to cotton wool pads.

The above-described cosmetic and/or the likewise already described kit can be used, according to the invention, for cosmetic treatment of the eye and of the skin, in particular for topical, cosmetic treatment of the skin in the region of the eye part, for topical, cosmetic treatment of the skin in the region of the lid, the upper lid, the lower lid, the tear sacs, the corners of the eye, the eyelashes, the glands of Moll, the glands of Zeis, the Meibomian glands, preferably for detaching dirt, encrusted and glutinous deposits in the region of the eyes.

The cosmetic is hereby applied preferably with the eye closed in the region of the eye area, in particular on the eyelid. It is thereby advantageous if the lid is heated to a temperature of 45° C. before applying the cosmetic by laying on a warming mask or a warm facecloth. As a result, any possible glutinous and thick secretion in the glands of Moll, Zeis and in the Meibomian glands, is made liquid and can be removed, for the large part, in fact by a lid massage simply with a finger. The movement during the lid massage should then be implemented in the direction of the exits of the Meibomian glands. For this purpose, the upper lid should be stroked several times with a finger from top to bottom and the lower lid several times from bottom to top. The liquified secretions can thus be detached and stroked out towards the inner corner of the lid.

The final cleaning and complete removal of the detached secretions, deposits and germs can then be effected with the help of a cotton wool pad saturated in the liquid. The same movements are thereby performed as already during the lid massage simply with a finger.

Likewise, the pads or sterile moist tissues saturated with the cosmetic can be placed below the warming eye mask on the closed eyelids or be inserted in an eye mask which is suitable for receiving the pad before laying on the closed eyes. The heat in combination with the treatment ingredients of the cosmetic effect softening and detaching of the encrusted and glutinous deposits. These can be subsequently removed easily with the pads or moist tissues and thus the closed eyes can be cleaned.

The present invention is explained in more detail with reference to the subsequent embodiments without restricting the invention hereto.

A cosmetic, given by way of example, formed from an aqueous composition which comprises, per litre, 0.20 g of sodium salt of hyaluronic acid, 1.00 g salicylic acid, 28.00 g sorbitol, 3.00 g Euphrasia mother tincture and citric acid and also sodium citrate as buffer system, is tested for storage life and stability. The pH value of this cosmetic was set to 7.0 to 7.2 with sodium hydroxide. The cosmetic comprises no emulsifiers and preservatives.

The composition of this cosmetic is reproduced in the subsequent table 1:

TABLE 1
Composition of the tested cosmetic

| | | |
|---|---|---|
| hyaluronic acid, sodium salt | 0.2 | g |
| salicylic acid | 1.0 | g |
| sorbitol | 28.0 | g |
| sodium citrate x 2 H2O | 8.5 | g |
| citric acid, water-free | 0.05 | g |
| Euphrasia mother tincture, DHU | 3.0 | g |

| | |
|---|---|
| sodium hydroxide | q.s. pH 7.0-7.2 |
| water f. 1. | ad 1,000 ml |

The storage life and stability of this cosmetic, given by way of example, was tested as described subsequently.

Samples of this cosmetic were stored at 25±2° C. and 60±5% relative atmospheric humidity for a period of time of 12 months or at 40° C. and 75% relative atmospheric humidity for a period of time of 6 months.

Stability and storage life are thereby established with respect to various properties of the cosmetic. What is crucial is appearance, smell, osmolality, pH value, particle freedom, viscosity, salicylic acid content, decomposition, accordance with UV-Vis spectra, spraying behaviour and volume of the aqueous solution.

The obtained results for storage at 25±2° C. and 60±5% atmospheric humidity are reproduced in the subsequent table 2.

“Corresponds” hereby means that the obtained measured results are within the tolerable ranges and hence the safety of the cosmetic demanded by cosmetic regulations for the consumer is ensured.

TABLE 2
Stability results at 25 ± 2° C. and 60 ± 5% relative atmospheric humidity

| Parameters | T0 | | 3 Mo | | 6 Mo | | 9 Mo | | 12 Mo | |
|---|---|---|---|---|---|---|---|---|---|---|
| appearance | clear, | | clear, | | clear, | | clear, | | clear, | |
| | yellowish | | yellowish | | yellowish | | yellowish | | yellowish | |
| | solution | | solution | | solution | | solution | | solution | |
| smell | of plants | | of plants | | of plants | | of plants | | of plants | |
| osmolality | 298 | mOsm/kg | 298 | mOsm/kg | 296 | mOsm/kg | 298 | mOsm/kg | 300 | mOsm/kg |
| pH value | 7.1* | | 7.1 | | 7.0 | | 7.0 | | 7.0 | |
| particle | corresponds | | corresponds | | corresponds | | corresponds | | corresponds | |
| freedom | | | | | | | | | | |
| viscosity | 1.3 | mm²/s | 1.4 | mm²/s | 1.3 | mm²/s | 1.3 | mm²/s | 1.4 | mm²/s |
| identity | corresponds | | corresponds | | corresponds | | corresponds | | corresponds | |
| salicylic acid | | | | | | | | | | |
| content | 98% | | 103% | | 99% | | 96% | | 99% | |
| salicylic acid | | | | | | | | | | |
| content | 0.98 | mg/ml | 1.03 | mg/ml | 0.99 | mg/ml | 0.96 | mg/ml | 0.99 | mg/ml |
| salicylic acid | | | | | | | | | | |

| decomposition | <0.1% | | 0.16% | <0.1% | <0.1% | 0.17% | |
|---|---|---|---|---|---|---|---|
| unknown | | | | | | | |
| individually | | | | | | | |
| decomposition | <0.1% | | 0.16% | <0.1% | <0.1% | 0.17% | |
| sum in total | | | | | | | |
| UV/VIS | beginning | | corresponds | corresponds | corresponds | corresponds | |
| spectrum | | | | | | | |
| sterility | corresponds | | — | — | — | corresponds | |
| average volume | 0.0058 | ml | — | — | — | 0.057 | ml |
| of one spray | | | | | | | |

| uniformity | corresponds | | — | — | — | corresponds |
|---|---|---|---|---|---|---|
| of the mass | | | | | | |
| of a spray | | | | | | |
| consistency | corresponds | | — | — | — | corresponds |
| pumping and | 3.0 | | — | — | — | — |
| spraying | corresponds | | | | | |
| behaviour | | | | | | |
| repriming and | — | | 1.0 | 1.0 | 1.0 | 1.0 |
| spraying | | | corresponds | corresponds | corresponds | corresponds |
| behaviour | | | | | | |
| rel. | beginning | | 0.10% | 0.19% | 0.29% | 0.39% |
| volume loss | | | | | | |
| remaining | 0.05 | ml | — | — | — | — |
| volume | | | | | | |
| bottle | | | | | | |

| | | | | | |
|---|---|---|---|---|---|
| appearance | corresponds | corresponds | corresponds | corresponds | corresponds |
| of the | | | | | |
| bottles | | | | | |

The obtained results for storage at 40±2° C. and 75±5% atmospheric humidity are reproduced in the following table 3.

“Corresponds” hereby means that the obtained measured results are within the tolerable ranges and hence the safety of the cosmetic demanded by cosmetic regulations for the consumer is ensured.

TABLE 3
Stability results at 40 ± 2° C. and 75 ± 5% relative atmospheric humidity

| | | | |
|---|---|---|---|
| Parameters | T0 | 3 Mo | 6 Mo |

| | | | |
|---|---|---|---|
| appearance | clear, | clear, | clear, |
| | yellowish | yellowish | yellowish |
| | solution | solution | solution |
| smell | of plants | of plants | of plants |

| | | | | | | |
|---|---|---|---|---|---|---|
| osmolality | 298 | mOsm/kg | 298 | mOsm/kg | 296 | mOsm/kg |

| | | | |
|---|---|---|---|
| pH value | 7.1 | 7.0 | 7.0 |
| particle | corresponds | corresponds | corresponds |
| freedom | | | |

| | | | | | | |
|---|---|---|---|---|---|---|
| viscosity | 1.3 | mm²/s | 1.4 | mm²/s | 1.4 | mm²/s |

| | | | |
|---|---|---|---|
| identity | corresponds | corresponds | corresponds |
| salicylic acid | | | |
| content | 98% | 103% | 99% |
| salicylic acid | | | |

| | | | | | | |
|---|---|---|---|---|---|---|
| content | 0.98 | mg/ml | 1.03 | mg/ml | 0.99 | mg/ml |
| salicylic acid | | | | | | |

| | | | |
|---|---|---|---|
| decomposition | <0.1% | 0.16% | <0.1% |
| unknown | | | |
| individually | | | |
| decomposition | <0.1% | 0.16% | <0.1% |
| sum in total | | | |
| UV/VIS | beginning | corresponds | corresponds |
| spectrum | | | |
| sterility | corresponds | — | corresponds |

| | | | | | |
|---|---|---|---|---|---|
| average volume | 0.0058 | ml | — | 0.057 | ml |
| of one spray | | | | | |

| | | | |
|---|---|---|---|
| uniformity | corresponds | — | corresponds |
| of the mass | | | |
| of a spray | | | |
| consistency | corresponds | — | corresponds |
| rel. | beginning | 0.19% | 0.36% |
| volume loss | | | |
| appearance of | corresponds | corresponds | corresponds |
| the bottles | | | |

The tests implemented respectively at an interval of 3 months show that the measured values for these above-mentioned values with the exception of the volume are identical both for storage at 25±2° C. and an atmospheric humidity of 60±5% and for storage at 40±2° C. and an atmospheric humidity of 75±5% within the scope of the measuring precision. The maximum established volume loss was likewise negligibly small at 0.39% within a period of time of 12 months.

The cosmetic mentioned above in the table can likewise be stored in a sterile manner in dosage devices without an infestation of microorganisms being recorded.

For this purpose, so-called “Tip Seal Integrity Tests” (TSIT) were implemented, with which it can be detected that the upper outlet valve of a dosage system which is used for storing the cosmetic protects the contents reliably from contaminations. In the course of the test, the dosage system is thereby actuated via the head several times whilst the dropper tip of the dosage system is immersed in a suspension of germs which comprises a high concentration of bacteria (10⁷ “colony forming units” per ml).

Subsequently, testing takes place after corresponding incubation as to whether the content of the dosage system is sterile. It was able to be established that even a very high concentration of germs around the outlet opening does not lead to contamination of the contained cosmetic.

With the cosmetic indicated above in table 1, likewise so-called “Closure and Ventilation Integrity Tests” (CVIT) were implemented. It is demonstrated by the CVIT that pump and container of the dosage system show a perfect fitting precision. It was established that the content of the dosage system itself is protected from aerosols of ultrasmall spores of germs, even under vacuum conditions. As a result of a series of rapid, successive actuations of the pump, a vacuum is generated in the system during exposure of the dosage system in the wet aerosol, with high density (100 CFU/cm²) of ultrasmall spores. Here also, the results show that, after corresponding incubation, contamination of the contained cosmetic does not result.

The previously represented test results show that the cosmetic according to the invention has not only excellent storage life but likewise a low tendency towards bacterial infestation.

1. A cosmetic for application on the eye and on the skin comprising or consisting of an aqueous solution which comprises hyaluronic acid or a salt of hyaluronic acid, salicylic acid or a salt of salicylic acid, sorbitol, Euphrasia mother tincture and a buffer system or consists hereof, characterised in that no emulsifiers are added to the solution.

2. The cosmetic according to claim 1, wherein the solution does not include any preservative, in particular no preservative selected from the group consisting of benzoic acid, propionic acid, hexadienoic acid, formaldehyde, inorganic sulphites, 2-phenoxyethanol, benzyl alcohol, hexamethylene tetramine, chloroacetamide, benzalkonium chloride, benzododecinium chloride, cetrimide, chlorohexidine, chlorobutanol, sodium hydroxymethylglycinate, sodium perborate, phosphonic acid, phenylmercuric acetate, polyhexanide, polyquad, purites, thiomersal.

3. The cosmetic according to claim 1, wherein the solution is phosphate-free.

4. The cosmetic according to claim 1, wherein the aqueous solution has a pH value in the range of 6.0 to 8.0, preferably in the range of 7.0 to 7.2.

5. The cosmetic according to claim 1, wherein the solution comprises salicylic acid or a salt of salicylic acid in a weight proportion of 0.01 to 2.00% by weight.

6. The cosmetic according to claim 1, wherein the solution comprises hyaluronic acid or a sodium salt of hyaluronic acid in a weight proportion of 0.01 to 1.00% by weight.

7. The cosmetic according to claim 1, wherein the solution comprises sorbitol in a weight proportion of 1.0 to 4.0% by weight.

8. The cosmetic according to claim 1, wherein the solution comprises Euphrasia mother tincture in a weight proportion of 0.001 to 0.4% by weight.

9. The cosmetic according to claim 1, wherein the solution comprises a buffer system, selected from the group consisting of citrate buffer, borate buffer, phosphate buffer, tris buffer, trometamol/maleic acid buffer system and also mixtures and combinations hereof.

10. The cosmetic according to claim 1, wherein the solution comprises a buffer system comprising or consisting of:
sodium citrate or a hydrate of sodium citrate,
citric acid and
NaOH.

11. The cosmetic according to claim 1, wherein the solution comprises a sodium salt of hyaluronic acid in a proportion of 0.01 to 1.0% by weight, salicylic acid in a proportion of 0.01 to 2.00% by weight, sorbitol in a proportion of 0.5 to 5.0% by weight, sodium citrate dihydrate in a proportion of 0.25 to 2.50% by weight and Euphrasia mother tincture in a proportion of 0.05 to 1.50% by weight, and has a pH value of 7.0 to 7.2.

12. A kit for cosmetic treatment of the eye, comprising a cosmetic according to claim 1 and also, separately from the cosmetic, at least a cotton wool pad, a cosmetic tissue and/or an eye mask.

13. The kit according to claim 12, wherein the eye mask is warmable and comprises a sodium acetate trihydrate-containing latent heat reservoir.

14. The kit according to claim 12, wherein the cotton wool pad, the paper tissue and/or the eye mask are configured to receive the cosmetic or the eye mask is configured to receive the cotton wool pad.

15. The kit according to claim 12, wherein the cosmetic is stored in a multi-dosage system, a disposable dosage system or in paper tissues which are moistened with the cosmetic.

16. A method of using the cosmetic according to claim 1, for cosmetic treatment of the eye and of the skin, in particular for topical, cosmetic treatment of the skin in the region of the eye area, for topical, cosmetic treatment of the skin in the region of the lid, the upper lid, the lower lid, the tear sacs, the corners of the eye, the eyelashes, the glands of Moll, the glands of Zeis, the Meibomian glands, for detaching dirt, encrusted deposits, glutinous deposits in the region of the eyes.

17. The method according to claim 16, wherein the cosmetic is applied with the eye closed in the region of the eye area, in particular on the eyelid.
